(19) United States
(12) Patent Application Publication
(10) Pub. No.: US 2003/0157071 A1
(43) Pub. Date: Aug. 21, 2003

(54) Treatment or replacement therapy using transgenic stem cells delivered to the gut

(76) Inventors: M. Michael Wolfe, Boston, MA (US); Lisa I. Jepeal, Billerica, MA (US); Michael O. Boylan, Milton, MA (US)

Correspondence Address: EDWARDS & ANGELL, LLP, P.O. BOX 9169, BOSTON, MA, 02209, US

(21) Appl. No.: 10/161,256

(22) Filed: May 31, 2002

Related U.S. Application Data
(60) Provisional application No. 60/294,772, filed on May 31, 2001.

(30) Foreign Application Priority Data
May 31, 2002 (WO) PCT/US02/17178

(51) Int. Cl. A61K 48/00; C12N 5/08

(52) U.S. Cl. 424/93.21; 435/372

(57) ABSTRACT

The present invention is directed to methods for hormone delivery to patients suffering from a condition associated with a hormone deficiency. The method involves transducing stem cells, such as bone marrow derived stem cells, with a hormone gene under the control of a cell-type specific promoter such as the glucose-responsive GIP promoter, such that the hormone gene is expressed only after the stem cells differentiate into the cells which express the cell-type specific promoter, and administering the stem cells to the patient. A preferred embodiment of the present invention is the use of GIP-insulin gene expression in K cells of the gut to treat diabetes.

### U.S. PATENT APPLICATION

This application for U.S. patent is filed as an utility application under U.S.C., Title 35, §111(a).

### RELATED U.S. PATENT APPLICATION

This application for U.S. patent relates and claims priority to U.S. provisional application, which was filed on May 31, 2001, is assigned provisional Serial No. 60/294,772 and is entitled Hormone Replacement Therapy using Transgenic Stem Cells Delivered to the Gut, and is incorporated herein by reference in its entirety.

### FIELD OF THE INVENTION

The present invention relates to transduced stem cells that can be delivered to the gut for treatment or replacement therapy, transduced stem cells attached to the gut, and methods. More specifically, the present invention is directed to treatment or replacement therapy by transducing derived stem cells with a gene encoding an active or other pharmaceutical agent, such as a protein, peptide, enzyme, hormone, hormone synthesis enzyme, pro-drug, precursor, etc., under the control of a tissue specific promoter. Preferably, the tissue-specific promoter is a gut-specific promoter, the glucose-dependent insulinotropic polypeptide (GIP) promoter.

### BACKGROUND

Many conditions are associated with a defect in the production of native peptide based and steroid hormones. For example, patients with type I and type II diabetes have insulin deficiencies, hypogonadism is associated with estrogen and/or testosterone deficiencies, a variety of reproductive disorders are associated with defects in luteinizing hormone (LH), follicular stimulating hormone (FSH), and prolactin, and obesity can be associated with leptin deficiencies.

A number of different approaches have been taken to treat individual hormone-deficient conditions and diseases. These approaches aim to supply the deficient hormone or hormone analog to the patient in a manner which mimics its delivery in healthy individuals. This is hard to do in practice because hormone production is highly regulated in vivo. Accordingly, mimicking such hormone delivery is one significant challenge of hormone replacement therapies.

Diabetes mellitus is a debilitating metabolic disease caused by absent (type I) or insufficient (type II) insulin production from pancreatic α cells. In these patients, glucose control depends on careful coordination of insulin doses, food intake, physical activity, and close monitoring of blood glucose concentrations. Ideal glucose levels are rarely attainable in patients requiring insulin injections. As a result, diabetic patients are presently still at risk for the development of serious long-term complications, such as cardiovascular disorders, kidney disease and blindness.

Another example of a hormone deficient condition is male hypogonadism, which is characterized by a deficiency of the steroid hormone testosterone. Male hypogonadism can be caused by disorders of the testes (primary), pituitary (secondary), or the hypothalamus (tertiary).^{1,8} Testosterone deficiency may occur as a result of Leydig cell dysfunction from primary disease of the testes, insufficient LH secretion from diseases of the pituitary, or insufficient GnRH secretion from the hypothalamus. Male hypogonadism has significant effects on the fertility, sexual function, and general health of patients.¹⁻⁸ Some causes of this disorder arc relatively common while others are rare. Klinefelter's syndrome, for example, occurs in about 1 in 500 men; it is a primary genetic disorder characterized by the presence of a second X chromosome (XXY) and is associated with a testicular abnormality that results in both androgen deficiency and irreversible infertility.⁹⁻¹¹

In men with clinical symptoms of primary or secondary hypogonadism, the testosterone deficiency can be treated with replacement therapy. However, successful fertility is improbable. Current formulations for androgen replacement therapy have significant problems. For example, pure oral testosterone is absorbed well in the gut but largely inactivated by the liver. Methyltestosterone, a synthetic testosterone, has a short half-life when administered orally or sublingually (2-3 hours) and is associated with hepatic toxicity, thus limiting its use. Furthermore. most clinical laboratories are unable to monitor adequate therapy by measurement of the steroid in the blood. Another synthetic testosterone, fluoxymesterone, has a longer hall life but significant hepatic toxicity. In addition, complications of androgen replacement therapy can include water retention, polycythemia, hypercalcemia, sleep apnea, prostate enlargement, and cardiovascular disease. Prolonged use of high doses of orally active androgens has been associated with a variety of peliosis hepatis, cholestatic jaundice, and hepatic neoplasms, including hepatic carcinoma. Peliosis hepatis can be a life-threatening or fatal complication. Pure testosterone is not known to produce these adverse effects.

Yet, another condition amenable to hormone replacement therapy is the treatment of certain cases of obesity by leptin. Body weight is determined by the competing balance of food intake and energy expenditure. A major advance in understanding the complex biological processes that regulate body weight was the identification of leptin, a protein hormone that is secreted by fat cells. Leptin plays a role in signaling to the brain to regulate food intake. Many obese individuals have defects in leptin, including defects in circulating leptin levels as well as resistance to leptin. One treatment for individuals with reduced levels of leptin is leptin replacement therapy. For individuals with resistance to leptin, recent advances have demonstrated that replacement therapy with human growth hormone (hGH) can make individuals more sensitive to leptin replacement therapy (when given in combination).

Traditional hormone replacement therapies have used a number of approaches. The standard treatment, for example for diabetes patients, is the repeated injection of the deficient hormone. In addition to being labor intensive, such injections can be associated with the introduction of foreign microbes, and hence potential infections.

Gene therapy has been proposed as an alternative approach for hormone replacement. Gene therapy uses a transgene (heterologous gene) to express the deficient hormone. It has been proposed as an attractive approach for hormone delivery because it offers the potential to overcome many of the problems in hormone delivery identified above. For example, because the patient expresses the hormone gene itself, the repeated insulin injection used by diabetics would be eliminated. Toxicity associated with synthetic hormones, such as testosterone analogs, would also eliminated. Indeed, the development of gene therapy approaches for hormone delivery is an area of intense research.

However, a significant number of challenges remain for gene therapy for hormone deficient conditions, including (1) effective delivery by the vector, (2) safety of the vector; (3) the ability to express the hormone transgene in an effective amount; (4) the ability to selectively target the desired cells by the vector; and (5) most importantly, the ability to coordinate the release of the transgenic hormone with the physiological demand for the hormone in the desired cells.

Accordingly, there is a definite need for methods in gene therapy to deliver an active or other pharmaceutical agent, such as a protein, peptide, enzyme, hormone, hormone synthesis enzyme, pro-drug, precursor, etc., to a patient suffering from a condition associated with an illness or deficiency. There is also a need in gene therapy to have regulated expression of the active or other pharmaceutical agent in response to physiological demand.

### SUMMARY OF THE INVENTION

We have now discovered a method for treating a patient having a condition, such as a hormone deficient condition like diabetes, which comprises administering to an animal, including a human, a population of stem cells transduced with a gene encoding an active or other pharmaceutical agent, such as a protein, peptide, enzyme, hormone, hormone synthesis enzyme, pro-drug, precursor, etc., that is under the control of a cell specific promoter. When the stem cells differentiate into cells expressing a certain cell type, the cell specific promoter will express the desired transgene (heterologous gene).

In accordance with the present invention, stem cells are transduced with a gene which encodes for any active or other pharmaceutical agent, such as a protein, peptide, enzyme, hormone, hormone synthesis enzyme, pro-drug, precursor, etc. Examples of such active or other pharmaceutical agents envisioned by the present invention include insulin, interferon, hormones, enzymes, somatostatin, anti-GIP, interleukins, chemokines, cytokines, EPO, nitiric oxide, synthetase, clotting factors, thrombin, pro-thrombin, etc.

In a preferred embodiment, the stem cells are transduced with a gene encoding a hormone or other active or pharmaceutical agent under the control of a K cell specific promoter. Preferably, the promoter is the glucose-responsive GIP promoter. Only those stem cells which differentiate into K cells of the gut will express the hormone.

In a further preferred embodiment to treat diabetes, the gene encoding insulin is under the control of the glucose-responsive GIP promoter, conferring glucosresponsive expression of insulin in the K cells of the gut.

Preferably, the stem cells are bone marrow derived stem cells, embryonic stem cells, cord blood cells, or stem cells derived from adipose tissue.

Preferably, the method of the present invention is used to treat patients with type I or type II diabetes (insulin), hypogonadism (estrogen, testosterone), reproductive disorders (LH, FSH, prolactin), obesity (leptin), infection, hormone deficiency, AIDS-diarrhea, IBS, GI bleeding, peptic ulcers, cancer, hepatitis, multiple sclerosis, melanoma, aging, erectile dysfunction, GI motility disorders, vascular tone, hypertension, etc.

Preferably, the stem cells are administered to the patient by infusion into the superior mesenteric artery or celiac artery, or by direct injection of stem cells into the internal mucosa in a pharmaceutically compatible excipient, such as a glucose solution or a physiological buffer or saline.

In one embodiment, the stem cells are also transduced with a “killer” gene under the control of an inducible promoter, such that the induction of the expression of the killer gene results in cell death of the cell expressing said gene. Preferably, the killer gene is the fas ligand, or encodes a toxic protein such as ricin, or is a gene encoding a fusion protein toxin based on Diphtheria toxin. safe and well tolerated.

These and other objects, features, and advantages of the present invention may be better understood and appreciated from the following detailed description of the embodiments thereof, selected for purposes of illustration and shown in the accompanying figures and examples. It should therefore be understood that the particular embodiments illustrating the present invention are exemplary only and not to be regarded as limitations of the present invention.

### BRIEF DESCRIPTION OF THE FIGS.

The foregoing and other objects, advantages and features of the invention, and the manner in which the same are accomplished, will become more readily apparent upon consideration of the following detailed description of the invention taken in conjunction with the accompanying figs., which illustrate preferred and exemplary embodiments, wherein:

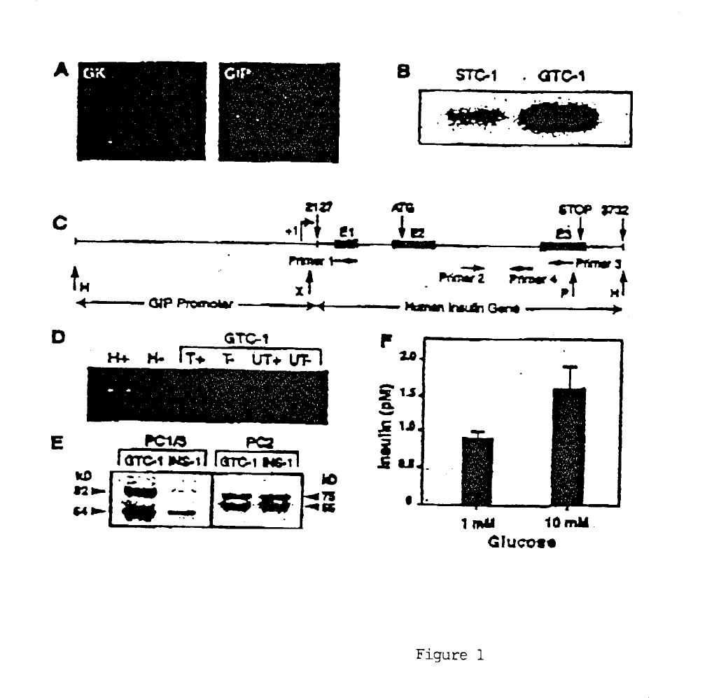

FIGS. 1A-F show expression of human insulin in tumor-derived GTC 1 cells.

FIG. 1A is a micrograph of immunofluorescence staining for glucokinase (GK, red) and GIP (green) in mouse duodenal sections.

FIG. 1B depicts Northern blot analysis of GIP MRNA in STC-1 and GTC-1 cells. K-cell enrichment was determined by comparing the amount of GIP mRNA in the parental cell line (STC-1) with that of the newly subcloned K-cell lines.

FIG. 1C is a schematic diagram of the plasmid (GIP/Ins) used for targeting human insulin expression to K cells. The rat GIP promoter (˜2.5 kb) was fused to the genomic human preproinsulin gene, which comprises 1.6 kb of the genomic sequence extending from nucleotides 2127 to 3732 including the native polyadenylation site. The three exons are denoted by filled boxes (E1, E2, and E3). The positions of primers used for RT-PCR detection of proinsulin mRNA are indicated. Hind III(H), Xho I(X), and Pvu II(P) sites are shown. Positions of start (ATG) and stop codons are indicated.

FIG. 1D shows RT-PCR analysis of cDNA from human islets (H) and GTC-1 cells either transfected (T) or untransfected (UT) with the GIP/Ins construct. Samples were prepared either in the presence (+) or absence (−) of reverse transcriptase.

FIG. 1E is a Western blot of proprotein convertases PC1/3 and PC2 expression in a (beta)-cell line (INS-1) and GTC-1 cell. Arrowheads indicate products at the predicted size for PC1/3 isoforms (64 and 82 kD) and PC2 isoforms (66 and 75 kD).

FIG. 1F is a graph depicting the effects of glucose on insulin secretion from GTC-1 cells stably transfected with the GIP/Ins construct. Triplicate wells of cells were incubated in media containing either 1 or 10 mM glucose (22). Medium was collected after 2 hours in each condition and assayed for human insulin. Values are means ±SEM; P<0.03.

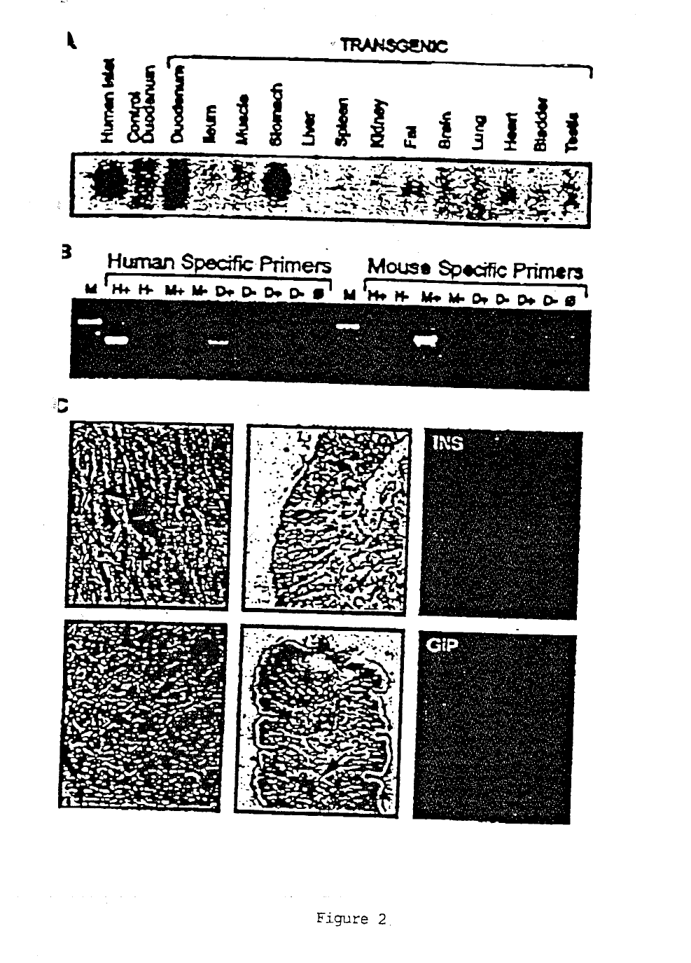

FIGS. 2A-C show targeted expression of human insulin to K cells in transgenic mice harboring the GIP/Ins transgene.

FIG. 2A depicts Northern blot analysis for human insulin gene expression in human islet, control mouse duodenum, and transgenic mouse tissues. The blot was probed with a 333-base pair cDNA fragment encompassing exons 1 and 2 and part of exon 3 of the human preproinsulin gene.

FIG. 2B shows RT-PCR analysis of cDNA from human islets (H), mouse islets (M), and duodenum samples (D) from two transgenic mice, with primers specific for human or mouse proinsulin. Samples were prepared either in the presence (+) or absence (−) of reverse transcriptase [phi], no DNA; M, markers.

FIG. 2C shows immunohistochemical staining for human insulin in sections of stomach (left column) and duodenum (middle column) from a transgenic mouse. Arrows indicate human insulin immunoreactive cells. Duodenal sections from the same animal were also examined by immunofluorescence microscopy (right column). Tissue sections were contained with antisera specific for insulin (INS, green) and G1P (red).

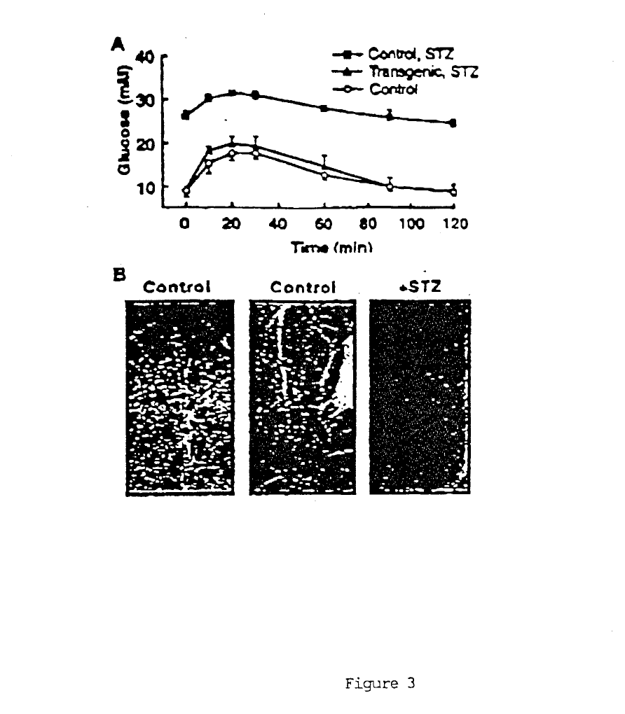

FIGS. 3A-B show production of human insulin from K cells protects transgenic mice froth diabetes induced by destruction of pancreatic [beta] cells.

FIG. 3A shows the results of oral glucose tolerance tests. Mice were given intraperitoneal injection of streptozotocin (STZ, 200 mg/kg), which destroys pancreatic beta cells, or an equal volume of saline. On the fifth day after treatment, after overnight food deprivation, glucose (1.5 g/kg body weight) was administered orally by feeding tube at 0 min. Results are means (±SEM) from at least three animals in each group.

FIG. 3B shows immunohistochemical staining for mouse insulin in pancreatic sections from control mice and an STZ-treated transgenic mouse. Arrows indicate mouse islets.

### DETAILED DESCRIPTION

By way of illustrating and providing a more complete appreciation of the present invention and many of the attendant advantages thereof, the following detailed description is given concerning the novel transduced stem cells, pharmaceuticals, and methods of manufacture and use, including methods useful for treatment or replacement therapy in hosts, such as animals including humans.

We have now discovered a method for selectively expressing a desired gene. Preferably, the desired gene encodes one or more active or other pharmaceutical agents, such as a protein, peptide, enzyme, hormone, hormone synthesis enzyme, pro-drug, precursor, etc., and can be used in hormone replacement therapy. The method comprises transducing stem cells with a desired gene such as one encoding an active or other pharmaceutical agent, such as a protein, peptide, enzyme, hormone, hormone synthesis enzyme, pro-drug, precursor, etc., under the control of a cell type specific promoter. When the stem cells differentiate into cells of the cell type that the promoter is specific to, the gene is expressed. This method involves administering by standard means, such as intravenous infusion or mucosal injection, the transduced stem cells to an as animal, including a human. Examples of active or other pharmaceutical agents contemplated by the present invention include insulin, interferon, hormones, enzymes, somatostatin, anti-GIP, interleukins, chemokines, cytokines, EPO, nitiric oxide, synthetase, clotting factors, thrombin, pro-thrombin, etc.

In a preferred embodiment, the present invention provides a method of treating diabetes by insulin replacement therapy. In this embodiment, stem cells are transduced with a hormone gene under the control of the K cell specific promoter, such as the GIP promoter. Only those cells which differentiate into K cells of the gut express the hormone.

Stem cells can be transduced ex vivo at high efficiency and by the appropriate selection of the cell-type specific promoter one can insure that the desired active or other pharmaceutical agent, such as a protein, peptide, enzyme, hormone, hormone synthesis enzyme, pro-drug, precursor, etc., e.g., insulin, is expressed by a desired cell type.

As used herein, a condition characterized by a hormone deficiency includes any condition associated with insufficient levels of an endogenous hormone. The present method can be used to treat a range of conditions, including those characterized by a hormone deficiency. Conditions (and the deficient hormone) include but are not limited to type I or type II diabetes (insulin), hypogonadism (estrogen, testosterone), reproductive disorders (LH, FSH, prolactin), or obesity (leptin).

According to one aspect of the invention, the stem cells are genetically altered prior to reintroducing the cells into the individual to introduce the gene encoding the deficient hormone or other agent in the individual. The present invention combines the use of a cell type specific promoter with the gene encoding a hormone to treat a patient deficient in that hormone. Thus, the selection of the cell type specific promoter depends on the hormone deficiency or other condition to be treated. Stem cells are capable of differentiating into numerous cell types. Furthermore, the differentiated cells should be capable of generating the agent, such as a hormone, such that it is accessible to its natural target population. For example, by secretion into the blood stream. Preferably, the cell type chosen is one which can naturally regulate the level of expression of the hormone.

The method of the present invention can use any promoter whose expression is regulated such that it is only expressed in a specific cell type. By using such promoters other cell types will not express the transgene because they do not allow expression of the regulated promoter. Preferably, the stem cells selected readily differentiate into the specific cell type desired.

For example, by using a K cell-specific promoter such as the glucose-dependent insulinotropic polypeptide (GIP) promoter expression of genes under control of the GIP promoter is limited to K cells of the gut. The GIP-promoter/hormone fusion gene will be expressed only in those cells that differentiate into K-cells, which will secrete the hormone into the blood stream. The GIP-promoter can be used with bone marrow derived stem cells, for example.

According to some aspects of the invention the stem cells may also be genetically altered to introduce an additional gene whose expression has therapeutic effect on the individual.

Stem cells include but are not limited to bone marrow derived stem cells, adipose derived stem cells, embryonic stem cells, and cord blood cells. Bone marrow derived stem cells refers to all stem cells derived from bone marrow; these include but are not limited to mesenchymal stem cells, bone marrow stromal cells, and hematopoietic stem cells. Bone marrow stem cells are also known as mesenchymal stem cells or bone marrow stromal stem cells, or simply stromal cells or stem cells. The stem cells of the present invention also include embryonic stem cells, stem cells derived from adipose tissue, uncultured unfractionated bone marrow stem cells, and cord blood cells.

The stem cells act as precursor cells which produce daughter cells that mature into differentiated cells. The stem cells can be from the individual in need of hormone replacement therapy or from another individual. Preferably, the individual is a matched individual to insure that rejection problems do not occur. Therapies to avoid rejection of foreign cells are known in the art. Accordingly, endogenous or stem cells from a matched donor may be administered by any known means, preferably intravenous injection, or injection directly into the appropriate tissue, to individuals suffering from a hormone deficient condition.

The discovery that isolated stem cells may be administered intravenously to replace a hormone missing in certain individuals provides the means for systemic administration. For example, bone marrow-derived stem cells may be isolated with relative ease and the isolated cells may be cultured to increase the number of cells available. Intravenous administration also affords ease, convenience and comfort at higher levels than other modes of administration. In certain applications, systemic administration by intravenous infusion is more effective overall. In a preferred embodiment, the stem cells are administered to an individual by infusion into the superior mesenteric artery or celiac artery. The stem cells may also be delivered locally by irrigation down the recipient's airway or by direct injection into the mucosa of the intestine.

In some aspects of the invention, individuals can be treated by supplementing, augmenting and/or replacing defective and/or damaged cells with cells that express the gene for the deficient hormone. The cells may be derived from stem cells of a normal matched donor or stem cells from the individual to be treated (i.e., autologous). By introducing normal genes in expressible form, individuals suffering from such a deficiency can be provided the means to compensate for genetic defects and eliminate, alleviate or reduce some or all of the symptoms.

A vector can be used for expression of the transgene encoding a desired wild type hormone or a gene encoding a desired mutant hormone. Preferably, the hormone gene is operably linked to regulatory sequences required to achieve expression of the gene in the stem cell or the cells that arise from the stem cells after they are infused into an individual. Such regulatory sequences include a promoter and a polyadenylation signal. The vector can contain any additional features compatible with expression in stem cells or their progeny, including for example selectable markers.

As used herein, the terms “transgene”, “heterologous gene”, “exogenous genetic material”, “exogenous gene” and “nucleotide sequence encoding the gene” are used interchangeably and meant to refer to genomic DNA, cDNA, synthetic DNA and RNA, mRNA and antisense DNA and RNA which is introduced into the stem cell. The exogenous genetic material may be heterologous or an additional copy or copies of genetic material normally found in the individual or animal. When cells are used as a component of a pharmaceutical composition in a method for treating human diseases, conditions or disorders, the exogenous genetic material that is used to transform the cells may encode proteins selected as therapeutics used to treat the individual and/or to make the cells more amenable to transplantation.

The regulatory elements necessary for gene expression include a promoter, an initiation codon, a stop codon. and a polyadenylation signal. It is necessary that these elements be operable in the stem cells or in cells that arise from the stem cells after infusion into an individual. Moreover, it is necessary that these elements be operably linked to the nucleotide sequence that encodes the protein such that the nucleotide sequence can be expressed in the stem cells and thus the protein can be produced. Initiation codons and stop codon are generally considered to be part of a nucleotide sequence that encodes the protein.

A variety of tissue-specific promoters, i.e. promoters that function in some tissues but not in others, can be used. Such promoters include GIP, EF2 responsive promoters, etc.

The effectiveness of some inducible promoters increases over time. In such cases one can enhance the effectiveness of such systems by inserting multiple repressors in tandem, e.g. TetR linked to a TetR by an IRES. Alternatively, one can wait at least 3 days before screening for the desired function. While some silencing may occur, given the large number of cells being used, preferably at least 1×10⁴, more preferably at least 1×10⁵, still more preferably at least 1×10⁶, and even more preferably at least 1×10⁷, the effect of silencing is minimal. One can enhance expression of desired proteins by known means to enhance the effectiveness of this system. For example, using the Woodchuck Hepatitis Virus Posttranscriptional Regulatory Element (WPRE). See Loeb, V. E., et al., Human Gene Therapy 10:2295-2305 (1999); Zufferey, R., et al., J. of Virol, 73:2886-2892 (1999); Donello, J. E., et al., J of Virol, 72:5085-5092 (1998).

Examples of polyadenylation signals useful to practice the present invention include but are not limited to human collagen I polyadenylation signal, human collagen II polyadenylation signal, and SV40 polyadenylation signal.

In order to maximize protein production, codons may be selected which are most efficiently transcribed in the cell. The skilled artisan can prepare such sequences using known techniques based upon the present disclosure.

The exogenous genetic material that includes the hormone gene operably linked to the tissue-specific regulatory elements may remain present in the cell as a functioning cytoplasmic molecule, a functioning episomal molecule or it may integrate into the cell's chromosomal DNA. Exogenous genetic material may be introduced into cells where it remains as separate genetic material in the form of a plasmid. Alternatively, linear DNA which can integrate into the chromosome may be introduced into the cell. When introducing DNA into the cell, reagents which promote DNA integration into chromosomes may be added. DNA sequences which are useful to promote integration may also be included in the DNA molecule. Alternatively, RNA may be introduced into the cell.

In another preferred embodiment, the transgene can be designed to induce selective cell death of the stem cells in certain contexts. In one example, the stem cells can be provided with a “killer gene” under the control of a tissue-specific promoter such that any stem cells which differentiate into cell types other than the desired cell type will be selectively destroyed. In this example, the killer gene would be under the control of a promoter whose expression did not overlap with the tissue-specific promoter.

Alternatively, the killer gene is under the control of an inducible promoter that would ensure that the killer gene is silent in patients unless the hormone replacement therapy is to be stopped. To stop the therapy, a pharmacological agent is added that induces expression of the killer gene, resulting in the death of all cells derived from the initial stem cells.

In another embodiment, the stern cells are provided with genes that encode a receptor that can be specifically targeted with a cytotoxic agent. An expressible form of a gene that can be used to induce selective cell death can be introduced into the cells. In such a system, cells expressing the protein encoded by the gene are susceptible to targeted killing under specific conditions or in the presence or absence of specific agents. For example, an expressible form of a herpes virus thymidine kinase (herpes tk) gene can be introduced into the cells and used to induce selective cell death. When the exogenous genetic material that inclines (herpes tk) gene is introduced into the individual, herpes tk will be produced. If it is desirable or necessary to kill the transplanted cells, the drug ganciclovir can be administered to the individual and that drug will cause the selective killing of any cell producing herpes tk. Thus, a system can be provided which allows for the selective destruction of transplanted cells.

Selectable markers can be used to monitor uptake of the desired gene. These marker genes can be under the control of any promoter or an inducible promoter. These are well known in the art and include genes that change the sensitivity of a cell to a stimulus such as a nutrient, an antibiotic, etc. Genes include those for neo, puro, tk, multiple drug resistance (MDR), etc. Other genes express proteins that can readily be screened for such as green fluorescent protein (GFP), blue fluorescent protein (BFP), luciferase, LacZ, nerve growth factor receptor (NGFR), etc.

For example, one can set up systems to screen stem cells automatically for the marker. In this way one can rapidly select transduced stem cells from non-transformed cells. For example, the resultant particles can be contacted with about one million cells. Even at transduction rates of 10-15% one will obtain 100-150,000 cells. An automatic sorter that screens and selects cells displaying the marker, e.g. GFP, can be used in the present method.

Vectors include chemical conjugates, plasmids, phage, etc. The vectors can be chromosomal, non-chromosomal or synthetic. Commercial expression vectors are well known in the art, for example pcDNA 3.1, pcDNA4 HisMax, pACH, pMT4, PND, etc. Preferred vectors include viral vectors, fusion proteins and chemical conjugates. Retroviral vectors include Moloney murine leukemia viruses and pseudotyped lentiviral vectors such as FIV or HIV cores with a heterologous envelope. Other vectors include pox vectors such as orthopox or avipox vectors, herpesvirus vectors such as a herpes simplex I virus (HSV) vector (Geller, A. I, et al., (1995), J. Neurochem, 64:487; Lim, F., ,et al., (1995) in DNA Cloning.—Mammalian Systems, D. Glover, Ed., Oxford Univ. Press, Oxford England; Geller, A. I., et al. (1993), Proc Natl. Acad. Sci.: U.S.A. 90:7603; Geller, A. I.,, et al., (1990), Proc Natl. Acad. Sci USA 87:1149), adenovirus vectors (1.eGal LaSalle et al. (1993), Science, 259:988: Davidson, et al. (1993) Nat. Genet 3: 219; Yang, et al., (1995) J Virol. 69:2004) and adeno-associated virus vectors (Kaplitt, M. G., et al, (1994) Nat. Genet. 8: 148).

As used herein, the introduction of DNA into a host cell is referred to as transduction, sometimes also known as transfection or infection.

The introduction of the gene into the stem cell can be by standard techniques, e.g. infection., transfection, transduction or transformation. Examples of modes of gene transfer include e.g., naked DNA, CaPO₄ precipitation, DEAE dextran, electroporation, protoplast fusion, lipofection, cell microinjection, and viral vectors, adjuvant-assisted DNA, gene gun, catheters, etc,

The vectors are used to transduce the stem cells ex vivo. One can rapidly select the transduced cell, by screening for the marker. Thereafter, one can take the transduced cells and grow them under the appropriate conditions or insert those cells into host animal.

As stated above, stem cells may also be derived from the individual to be treated or a matched donor. Those having ordinary skill in the art can readily identify matched donors using standard techniques and criteria.

Two preferred embodiments provide bone marrow or adipose tissue derived stem cells, which may be obtained by removing bone marrow cells or fat cells, from a donor, either self car matched, arid placing the cells in a sterile container with a plastic surface or other appropriate surface that the cells come into contact with. The stromal cells will adhere to the plastic surface within 30 minutes to about 6 hours. After at least 30 minutes, preferably about four hours. the non-adhered cells may be removed and discarded. The adhered cells are stem cells which are initially non-dividing. After about 2-4 days however the cells begin to proliferate.

According; to preferred embodiments, stem cells are cultured in medium supplemented with 2-20% fetal calf serum or serum-free medium with or without additional supplements. Preferably. stem cells are cultured in 10% fetal calf serum in DMEM. Culture medium is replaced every 2-3 days.

After isolating the stem cells, the cells can be administered upon isolation or after they have been cultured. Isolated stem cells administered upon isolation are administered within about one hour after isolation. Generally, stem cells may be administered immediately upon isolation in situations in which the donor is large and the recipient is an infant. It is preferred that stem cells are cultured prior to administrations. Isolated stem cells cart be, cultured from 1 hour to over a year. In some preferred embodiments, the isolated stem cells are cultured prior to administration for a period of time sufficient to allow them to convert from non-cycling to replicating cells. Preferably the cells are cultured for 3-30 days, more preferably 4-14 days, still more preferably 5-10 days, most preferably 7 days.

In a preferred embodiment, stem cells can be cultured for 7 days before administration. The stem, cells can be either 1) isolated, non-cycling stem cells that are first transfected and then administered as non-cycling cells, 2) isolated, non-cycling stem cells that are first transfected, then cultured for a period of time sufficient to convert from non-cycling to replicating cells, and then administered, 3) isolated, non-cycling stem cells that are first cultured for a period of time sufficient to convert from non-cycling to replicating cells, then transfected, and then administered, or 4) isolated, non-cycling stem cells are first cultured for a period of time sufficient to convert from non-cycling to replicating cells, then transfected, then cultured and administered.

For administration of stem cells, the isolated stem cells are removed from culture dishes, washed with saline, centrifuged to a pellet and resuspended in, for example, a glucose solution or a physiological buffer or saline compatible with the stem cells, which are infused into the patient.

Between 10⁵ and 10¹³ cells per 100 kg person are administered per by infusion. Preferably, between about 1-5×10⁸ and 1-5×10¹² cells are infused intravenously per 100 kg person. More preferably, between about 1×10⁹ and 5×10¹¹ cells are infused intravenously per 100 kg person. For example, dosages such as 4×10⁹ cells per 100 kg person and 2×10¹¹ cells can be infused per 100 kg person. The cells can also be injected directly into the intestinal mucosa through an endoscope.

In some embodiments, a single administration of cells is provided. In other embodiments, multiple administrations would be used. Multiple administrations can be provided over periodic time periods such as an initial treatment regime of 3-7 consecutive days, and then repeated at other times.

In some embodiments, fresh bone marrow or adipose tissue cars be fractionated using fluorescence activated call sorting (FACS) with unique cell surface antigens to isolate specific subtypes of stem cells (such as bone marrow or adipose derived stem cells) for injection into recipients either directly (without culturing) or following culturing, as described above.

A GIP-GFP transgenic mouse can be generated and used to develop strategies to optimize the delivery of GIP-hormone transduced stem cells. The transgenic mice can be used as a source of embryonic and adult stem cells. In order for a stem cell mediated approach to be operable, two requirements must be met: 1) the stem cells delivered to the intestine must survive; and 2) a certain percentage of the engrafted stem cells must differentiate into K-cells. To address the first point, transgenic lines can be generated in the context of the ROSA mouse. This mouse contains the lazZ under the control of a non-specific constitutive promoter, and allows identification of all cells derived from this mouse by assaying for beta-galactosidase. Therefore, survival of implanted stem cells can be monitored by the expression of beta-galactosidase, while the differentiation can be monitored by the expression of GFP.

In addition, the GIP-GFP transgenic mouse can be used as a source of purified K-cells. The presence of GFP in K-cells permits the identification and selection of these cells by fluorescence-activated cell sorting (FACS). RNA can be isolated from purified K-cells and subjected to microarray analysis. Information obtained from the microarray analysis can provide a better understanding of the type of genes that are activated when intestinal stem cells differentiate into K-cells.

A GIP-GFP chimeric gene has been constructed in the Wolfe laboratory. This gene consists of approximately 2.5 kilobase pairs of the GIP 5 flanking region fused to the gene encoding the green fluorescent protein (GFP). The cloning vector used was pEGFP. The GIP-GFP gene can be excised from the cloning vector, and the DNA can be purified and injected into the pronuclei of fertilized mouse eggs. The fertilized eggs will be transplanted into the uterus of pseudopregnant mice. Resulting offspring can be screened for the presence of the intact transgene in their genomes, using a combination of the polymerase chain reaction and Southern blot hybridization. Offspring containing the intact GIP-GFP gene (GIP-GFP⁺/GIP-GFP⁻) will then be bred with syngeneic animals (GIP-GFP⁺/GIP-GFP⁻). Heterozygous GIP-GFP⁺/GIP-GFP⁻ offspring that contain GFP in their intestinal K-cells will be in-bred to produce homozygous GIP-GFP⁺/GIP-GFP⁻ mice.

Introduction of GIP-GFP⁺ Stem Cells into a Host. Once it has been demonstrated that engrafted stem cells can survive in the intestine and differentiate into K-cells, a method for efficiently transducing stem cells in vitro can be developed. To optimize the transduction process, embryonic and adult stem cells are isolated from transgenic ROSA mice and transduced with the GIP-GFP gene. After isolation, stem cells can be grown on various supports and in various media to determine the best conditions for stem cell growth and transduction. Care will be taken to ensure that conditions do not promote the differentiation of these cells in vitro. Electroporation can be used to transduce the cells. A drug resistant gene such as neomycin can be included with the GIP-GFP DNA to enable the selection of transduced cells. Transduced cells can then be introduced by injection into the intestinal mucosa of syngeneic hosts. At various times after injection, animals can be sacrificed and their intestines examined for the presence of beta-galactosidase expression and for GFP expression. Expression of beta-galactosidase indicates survival of injected stem cells, and the expression of GFP indicates the differentiation of these stem cells into K-cells. Isolation, growth and transduction of stem cells can be optimized to generate the greatest survival of engrafted cells along with the highest percentage of these cells differentiating into K-cells.

The following example is given by way of illustration only and is not to be considered a limitation of this invention or many apparent variations of which are possible without departing from the spirit or scope thereof.

### EXAMPLE

Materials and Methods

The rat GIP promoter was obtained from a rat genomic [lambda] DASH library (Stratagene. La Jolla, Calif.) by plaque hybridization with the rat GIP cDNA clone as described previously [M. O. Boylan et al., J. Biol. Chem. 273, 17438 (1997)]. The GIP promoter was subcloned into the promoterless pEGFP-I plasmid (Clontech, Palo Alto, Calif.). The resulting reporter vector was transfected into STC-1 cells (gift from D. Drucker, University of Toronto) using LipofectAMINE reagent (GIBCO BRL/Life Technologies, Rockville, Md.). Cells were dispersed with trypsin/EDTA, and fluorescent cells expressing EGFP were doubly hand-picked and placed into individual dishes for clonal expansion.

Total RNA from GTC-1 and STC-1 cells was isolated with Trizol (GIBCO) according to manufacturer's instructions. Total cell RNA (20 μg from each sample) was electrophoretically separated and transferred to nylon membrane. Hybridization was performed with the radiolabeled 660-bp Eco RI fragment of the rat GIP cDNA that was random-primed with [[alpha]-³² P]deoxycytidine 5′-triphosphate (dCTP). After hybridization, membranes were washed and exposed to x-ray film.

Reverse transcription-PCR analysis was used to determine whether the preproinsulin gene is appropriately transcribed and processed in transfected cells. Total RNA was isolated with Trizol. Total RNA (5 μg) isolated from transfected and nontransfeeted cells and human islets was reverse-transcribed with oligo(dT) primer by using superscript II reverse transcriptase (GEBCO). The cDNA product (2 μl) was then amplified with human preproinsulin gene-specific primers (primers 1 and 3, FIG. 1C).

Cells were lysed in ice-cold radioimmunoprecipitation assay buffer and supernatants were assayed for total protein content by using the Bradford method [I M. Bradford, Anal. Biochem. 72, 248 (1976)]. Cell lysate protein (50 μg) was fractionated on 10% SDS-polyacrylamide gel electrophoresis. After gel separation, proteins were electroblotted onto nitrocellulose membranes and incubated with polyelonal antibodies that recognize PC1/3 and PC2 (provided by I. Lindberg, Louisiana State Medical Center). Membranes were washed and then incubated with goat antiserum to rabbit coupled to horseradish peroxidase (Amersham Pharmacia Biotech, Uppsala, Sweden). The blots were then developed with a chemiluminescence Western blotting detection kit.

GTC-1 cells grown to 70 to 80% confluence in 12-well plates were given restricted nutrients for 2 hours in Dulbecco's minimum essential medium (DMEM) with 1.0 mM glucose and 1% fetal calf serum (FCS). Cells were washed and then incubated in 0.5 ml of release media (DMEM plus 1% FCS with either 1.0 or 10.0 mM of glucose) for 2 hours. Insulin levels in media were measured using the human-specific insulin ELISA kit [American Laboratory Products Company (ALPCO), Windham, N. H.] according to supplier's instructions.

The GIP/Ins fragment (4.2 kb) was excised with Hind III and gel-purified. Transgenic mice were generated by pronuclear microinjection of the purified transgene into fertilized embryos that were then implanted into pseudopregnant females. Transgenic mice were identified by Southern blot analysis. Ear sections were digested. and the purified DNA was cut with Xho I and PvuII (FIG. 1C), electrophoretically separated, and transferred to nylon membrane. For the detection of the transgene, a 416-bp human insulin gene fragment encompassing intron 2 was amplified by using primers 2 and 4 (FIG. 1C). The PCR product was prepared as a probe by radiolabeling with [[alpha]³²P]dCTP, and bands were detected by autoradiographv. Southern analysis results were further confirmed by PCR amplification of the genomic DNA using primers 2 and 4. Positive founders were outbred with wild-type FVB/N mice to establish transgenic lines.

Primers used were human proinsulin-specific, forward 5′-CCAGCCGCAGCCTTTGTFA-3′ and reverse 5′-GGTACAGCATTGTTCCACAATG-3′; mouse proinsulin-specific, forward 5′-ACCACCAGCCCTAAGTGAT-3′ and reverse 5′-CTAGTTGCAGTAGTTCTCCAGC-3′. PCR conditions were as follows: denaturation at 94° C. for 1 min, annealing at 50° C. for 1 min, and extension at 72° C. for 1 min for 45 cycles: PCR products were analyzed on a 2% agarose gel and visualized by ethidium bromide staining. The human-and mouse-specific primer sets yield 350-bp and 396-bp products, respectively.

Tissues were fixed in Bouin's solution overnight and embedded in paraffin. Tissue sections 5 μm thick were mounted on glass slides. For inununohistochemistry, the avidin-biotin complex method was used with peroxidase and diaminobenzidine as the chromogen. Sections were incubated with guinea pig antibody to insulin (1:500, Lineo Research. St. Charles, Mo.) or mouse antibody to GIP (1:200, a gift from R. Pederson, University of British Columbia) for 30 min and appropriate secondary antibodies for 20 min at room temperature. Biotinylated secondary antibodies were used for immunohistochemistry, and fluorescein- or Cy3-conjugated secondary antibodies were used for immunotluorescence.

Plasma insulin levels were measured using the ultrasensitive human-specific insulin ELISA kit (ALPCO) according to supplier's instructions. This assay has <0.01% cross-reactivity with human proinsulin and C peptide and does not detect mouse insulin. Plasma C-peptide measurements were made with a rat/mouse C-peptide radioimmunoassay kit (Linco Research). The assay displays no cross-reactivity with human C peptide..

Streptozotocin was administered to 8-week-old transgenic and age-matched control mice via an intraperitoneal injection at a dose of 200 mg/kg body weight in citrate buffer. At this high dose of streptozotocin, mice typically display glucosuria within 3 days after injection. For oral glucose tolerance tests, glucose was administered orally by feeding tube (1.5 g/kg body weight) as a 50% solution (w/v) to mice that had been without food for 14 hours. Blood samples (40 μl) were collected from the tail vein of conscious mice at 0, 10, 20, 30, 60, 90, and 120 min after the glucose load. Plasma glucose levels were determined by enzymatic, colorimetric assay (Sigma), and plasma insulin levels were measured using the ultrasensitive human-specific insulin BLISA kit (27).

Pancreata were homogenized and then sonicated at 4° C. in 2 mM acetic acid containing 0.25% bovine serum albumin. After incubation for 2 hours on ice, tissue homogenates were resonicated and centrifuged (8000 g, 20 min), and supernatants were assayed for insulin by radioimmunoassay.

To measure total insulin in the pancreas, pancreata were homogenized and then sonicated at 4° C. in 2 mM acetic acid containing 0.25% bovine serum albumin. After incubation for 2 hours on ice, tissue homogenates were resonicated and centrifuged (8000 g, 20 min), and supernatants were assayed for insulin by radioimmunoassay.

The present invention provides a method for genetic engineering of non-[beta] cells to release insulin upon feeding as a therapeutic modality for patients with diabetes. A tumor-derived K-cell line was induced to produce human insulin by providing the cells with the human insulin gene linked to the 5′-regulatory region of the gene encoding glucose-dependent insulinotropic polypeptide (GTp). Mice expressing this transgene produced human insulin specifically in gut K cells. This insulin protected the mice from developing diabetes and maintained glucose tolerance after destruction of the native insulin-producing [beta] cells.

Diabetes mellitus (DM) is a debilitating metabolic disease caused by absent (type 1) or insufficient (type 2) insulin production from pancreatic [beta] cells. In these patients, glucose control depends on careful coordination of insulin doses, food intake, and physical activity and close monitoring of blood glucose concentrations. Ideal glucose levels are rarely attainable in patients requiring insulin injections (1). As a result, diabetic patients are presently still at risk for the development of serious long-term complications, such as cardiovascular disorders, kidney disease, and blindness.

A number of studies have addressed the feasibility of in vivo gene therapy for the delivery of insulin to diabetic patients. Engineering of ectopic insulin production and secretion in autologous non-[beta] cells is expected to create cells that evade immune destruction and to provide a steady supply of insulin. Target tissues tested include liver, muscle, pituitary, hematopoietic stem cells, fibroblasts, and exocrine glands of the gastrointestinal tract (2-7). However, achieving glucose-dependent insulin release continues to limit the clinical application of these approaches. Some researchers have attempted to derive glucose-regulated insulin production by driving insulin gene expression with various glucose-sensitive promoter elements (8). However, the slow time course of transcriptional control by glucose makes synchronizing insulin production with the periodic fluctuations in blood glucose levels an extremely difficult task. The timing of insulin delivery is crucial for optimal regulation of glucose homeostasis; late delivery of insulin can lead to impaired glucose tolerance and potentially lethal episodes of hypoglycemic shock. Therefore, what is needed for insulin gene therapy is a target endocrine cell that is capable of processing and storing insulin and of releasing it in such a way that normal glucose homeostasis is maintained.

Other than beta cells, there are very few glucose-responsive native endocrine cells in the body. K cells located primarily in the stomach, duodenum, and jejunum secrete the hormone GIP (9. 10), which normally functions to potentiate insulin release after a meal (11). Notably, the secretion kinetics of GIP in humans closely parallels that of insulin, rising within a few minutes after glucose ingestion and returning to basal levels within 2 hours (12). GIP expression (13) and release (14) have also been shown to be glucose-dependent in vitro. However, the mechanism that governs such glucose-responsiveness is unclear. We made an interesting observation of glucokinase (GK) expression in gut K cells (FIG. 1A). GK, a rate-limiting enzyme of glucose metabolism in [beta] cells, is recognized as the pancreatic “glucose-sensor” (15). This observation raises the possibility that GK may also confer glucose-responsiveness to these gut endocrine cells. Given the similarities between K cells and pancreatic [beta] cells, we proposed to use K cells in the gut as target cells for insulin gene therapy.

A GIP-expressing cell line was established to investigate whether the GIP promoter is effective in targeting insulin gene expression to K cells. This cell line was cloned from the murine intestinal cell line STC-1, a mixed population of gut endocrine cells (16). K cells in this population were visually identified by transfection of an expression plasmid containing ˜2.5 kb of the rat GIP promoter fused to the gene encoding the enhanced green fluorescent protein (EGFP). After clonal expansion of the transiently fluorescent cells, clones were analyzed for the expression of GIP MRNA by Northern blotting. The amount of GIP mRNA in one clone (GIP tumor cells; GTC-1) was ˜8 times that in the parental heterogeneous STC-1 cells (FIG. 1B). Transfection of GTC-1 cells with the human genomic preproinsulin gene linked to the 3′ end of ˜2.5 kb of the rat GIP promoter (FIG. 1C, GIP/Ins) resulted in a correctly processed human preproinsulin mRNA transcript (FIG. 1D). When the same GIP/ins construct was transfected into a [beta]-cell line (INS-1), a liver cell line (HepG2), and a rat fibroblast (3T3-L1) cell line, little human preproinsulin mRNA was detectable (17). These observations suggest that the GIP promoter used is cell-specific and is likely to be effective in targeting transgene expression specifically to K cells in vivo. Western blot analysis revealed that the proprotein convertases required for correct processing of proinsulin to mature insulin (PC1/3 and PC2) (18) were expressed in GTC-1 cells (FIG. 1F). Consistent with this observation, a similar molar ratio of human insulin and C peptide was observed in culture medium from cells transfected with the GIP/Ins construct. Furthermore, release of insulin from these cells was glucose-dependent t (FIG. 1F).

To determine whether the GIP/Ins transgene can specifically target expression of human insulin to gut K cells in vivo, we generated transgenic mice by injecting the linearized GIP/Ins fragment into pronuclei of fertilized mouse embryos. In the resulting transgenic mice, human insulin was expressed in duodenum and stomach, but not in other tissues examined (FIG. 2B). The insulin mRNA detected in the duodenum; sample from the transgenic mice was confirmed by reverse transcription-polymerase chain reaction (RT-PCR) to be a product of the transgene and not contamination from adjacent mouse pancreas (FIG. 2B). This tissue distribution of insulin gene expression in transgenic animals corresponds to the known tissue expression pattern of GIP (9.10). The cellular localization of human insulin protein was determined in tissue samples from transgenic mice by using antisera to human insulin. Insulin immunoreactivity was detected in distinct endocrine cells in sections from stomach (FIG. 2C, left) and duodenum (FIG. 2C, middle) of transgenic animals. Furthermore, these cells were identified as K cells by the coexpression of immunoreactive GIP (FIG. 2C, right), confirming that human insulin production was specifically targeted to gut K cells. Plasma levels of human insulin in pooled samples collected after an oral glucose challenge were 39.0±9.8 pM (n=10, mean±SEM) in transgenie and undetectable in controls (n=5). It is interesting that amounts of mouse C peptide after an oral glucose load in transgenics were ˜30% lower than those of controls (227,1±31.5 pM versus 361.5±31.2 pM, n=3 in each group, mean±SEM). This observation suggests that human insulin produced from the gut may have led to compensatory down-regulation of endogenous insulin production.

Whether human insulin production from gut K cells was capable of protecting transgenic mice from diabetes was investigated. Streptozotocin (STZ), a [beta]-cell toxin, was administered to transgenic mice and age-matched controls. In control animals, STZ treatment resulted in fasting hyperglycemia (26.2±1.52 mM, n=3, mean±SEM) and the presence of glucose in the urine within 3 to 4 days, indicating the development of diabetes. When left untreated, these animals deteriorated rapidly and died within 7 to 10 days. In contrast. neither glucosuria nor fasting hyperglycemia (9.52±0.67 mM, n=5, mean±SEM) was detected in transgenic mice for up to 3 months after STZ treatment, and they continued to gain weight normally. To determine whether insulin production from K cells was able to maintain oral glucose tolerance in these mice, despite the severe [beta]-cell damage by STZ, mice were challenged with an oral glucose load. Control mice given STZ were severely hyperglycemic both before and after the glucose ingestion (FIG. 3A). In contrast, STZ-treated transgenic mice had normal blood glucose levels and rapidly disposed of the oral glucose load as did normal age-matched control mice (FIG. 3A). To ensure that the STZ treatment effectively destroyed the [beta] cells in these experimental animals, pancreatic sections from controls and STZ-treated transgenic animals were immunostained for mouse insulin. The number of cell clusters positively stained for mouse insulin was substantially lower in STZ-treated animals when compared with sham-treated controls (FIG. 3B). Total insulin in the pancreas in STZ-treated transgenic mice was only 0.5% that of the sham-treated controls (0.18 versus 34 μg insulin per pancreas, n=2). These STZ-treated transgenic mice disposed of oral glucose in the same way that normal mice do, despite having virtually no pancreatic [beta] cells, which indicates that human insulin produced from the gut was sufficient to maintain normal glucose tolerance. Previous attempts to replace insulin by gene therapy prevented glucosuria and lethal consequences of diabetes, such as ketoacidosis, but were unable to restore normal glucose tolerance (2). Our findings suggest that insulin production from gut K cells may correct diabetes to the extent of restoring normal glucose tolerance.

The identification of a glucose-responsive endocrine cell target for endogenous insulin production represents an important step toward a potential gene therapy for DM. However, an effective means of therapeutic gene delivery to gastrointestinal cells needs to be developed. There are many features of the upper gastrointestinal tract that make it an attractive target tissue for gene therapy. This region of the gut is readily accessible by noninvasive techniques, such as oral formulations or endoscopic procedures, for therapeutic gene transfer. The gut epithelium is also one of the most rapidly renewing tissues in the body and has a large number of proliferative cells, thus allowing the deployment of retroviral vectors that are approved for human investigation. Indeed, the gut—the largest endocrine organ—may have the highest concentration of stem cells found anywhere in the body (19). These cells, which give rise to the various cells lining the gut epithelium, including billions of K cells (20), are situated in the crypts of LieberkUhn (19). Successful transduction of these stem cells should allow long-term expression of the transgene, as occurs in our transgenic mice. Viral vectors have already been developed that deliver genes to cells of the intestinal tract, including the stem cells (21-22). Given the massive number of K cells, appropriately regulated insulin secretion from a fraction of these cells maw be sufficient for adequate insulin replacement for patients with diabetes. This gene therapy approach is also amenable to the expression of alternate insulin analogs, which could have more potent glucose-lowering activity and/or longer duration of action, as required. Therefore, genetic engineering of gut K cells to secrete insulin may represent a viable mode of therapy for diabetes, freeing patients from repeated insulin injections and reducing or even eliminating the associated debilitating complications.

References

1. U.K. Prospective Diabetes Study (UKPDS) Group, Lancet 352. 837 (1998).

2. T. M. Kolodka, M. Finegold, L. Moss, S. L. C. Woo, Proc. Natl. Acad. Sci. U.S.A. 92, 3293 (1995).

3. R. J. Bartlett et al., Transplant. Proc. 29, 2199 (1997).

4. M. A. Lipes et al., Proc. Natl. Acad. Sci. U S.A. 93, 8595 (1996).

5. M. R. Bochan, R. Shah, R. A. Sidner, R. M. Jindal, Transplant. Proc. 31, 690 (1999).

6. L. Falqui et al., Hum. Gene Ther. 10,1753 (1999).

7. 1. D. Goldfine et al., Nature Riotechnol. 15, 1378 (1997).

8. P. M. Thule, J. Liu, L. S. Philips, Gene Ther. 7, 205 (2000).

9. G. L. Ferri et al., Gastroenterology 85, 777 (1983).

10. C. M. Yeung, C. K. C. Wong, S. K. Chung, S. S. M. Chung, B. K. C. Chow, Mol. Cell. Endocrinol. 154, 161 (1999).

11. M. M. Wolfe, M. O. Boylan, T. J. Kieffer, C. C. Tseng, in Gastrointestinal Endocrinology, G. H. Greeley Jr., Ed. (Humana Press, Totowa, N.J., 1999), vol. 8, p. 439.

12. S. Cataland, S. E. Crockett, J. C. Broom, E. L. Mazzaferri, J. Clin. Endocrinol. Metab. 39, 223 (1974).

13. C. C. Tseng, L. A. Jarboe, M. M. Wolfe, Am. J. Physiol. 266, 6887 (1994).

14. T. J. Kieffer, A. M. J. Buchan, H. Barker, J. C. Brown, R. A, Pederson, Am. J. Physiol. 267, E489 (1994).

15. F. M. Matschinsky, B. Glaser, M. A. Magnuson, Diabetes 47, 307 (1998).

16. G. Rindi et al., Am. J. Pathol. 136, 1349 (1990).

17. A, T. Cheune et al., unpublished data.

18. D. F. Steiner, Curr. Opin. Chem. Biol. 2, 31 (1998).

19. P. B. Simon, A. G. Rcnehan, C. S. Potten, Carcinogenesis 21, 469 (2000).

20. O. Sandstr6m, M. El-Salhy, Mech. Ageing Dev. 108, 39 (1999).

21. M. A. Croyle, M. Stone, G. L. Amidon, B. J. Roessler, Gene Ther. 5, 645 (1998).

22. S. J. Henning, Adv. Drug Deliv. Rev. 17, 341 (1997).

All references described or cited herein are incorporated herein by reference in their entireties.

Accordingly, it will be understood that embodiments of the present invention have been disclosed by way of example and that other modifications and alterations may occur to those skilled in the art without departing from the scope and spirit of the appended claims. Thus, the invention described herein extends to all such modifications and variations as will be apparent to the reader skilled in the art, and also extends to combinations and sub-combinations of the features of this description and the accompanying Figs.

It will also be understood that, although preferred embodiments of the present invention have been illustrated in the accompanying Figs. and described in the foregoing detailed description and example, the invention is not limited to the embodiments disclosed, but is capable of numerous rearrangements, modifications and substitutions without departing from the spirit of the invention as set forth and defined by the following claims.

Having described our invention, we claim:

1. A method for selectively expressing a desired hormone in a host for treating or replacing hormone in the host, said method comprising:
(a) transducing a population of stem cells with a DNA sequence containing a gene encoding said desired hormone or a gene encoding a synthetic enzyme for said hormone, wherein said gene is operably-linked to a cell type specific promoter;
(b) administering said transduced stem cells to a host under conditions wherein at least some of said stem cells differentiate into cells of the type said cell type specific promoter is specific for (referred to, as differentiated stem cells); and
(c) allowing said differentiated stem cells to express said desired hormone or a synthetic enzyme for said hormone in the host to treat or replace hormone in the host.

2. The method of claim 1, wherein said host has a hormone deficiency condition.

3. The method of claim 1, wherein the stem cells are selected from the group consisting of bone marrow derived stem cells, embryonic stem cells, adipose tissue derived stem cells, and cord blood cells.

4. The method of claim 2, wherein the condition is selected from the group consisting of type I diabetes, type II diabetes, hypogonadism, reproductive disorders, and obesity.

5. The method of claim 1, wherein the hormone gene is selected from the group consisting of insulin, estrogen, testosterone. luteinizing hormone, follicle stimulating hormone, prolactin, leptin, and angiotensin.

6. The method of claim 1, wherein the tissue specific promoter is glucose-dependent insulinotropic polypeptide (GIP).

7. The method of claim 6, wherein the stem cells differentiate into K cells of the out.

8. The method of claim 1, wherein the stem cells are administered to the host by infusion into the superior mesenteric artery or celiac artery.

9. The method of claim 1 wherein the stem cells are further transduced with a killer gene under the control of a regulatable promoter, wherein the induction of the expression of the killer gene results in cell death of the cell expressing said gene.

10. The method of claim 9, wherein the killer gene is the fas ligand.

11. The method of claim 1, wherein the stem cells are administered to the host by injection into the intestinal mucosa.

12. A method for selectively expressing a desired active or pharmaceutical agent comprising:
(a) transducing a population of stem cells with a DNA sequence containing a gene encoding said desired active or pharmaceutical agent, wherein said gene is operably-linked to a cell type specific promoter;
(b) administering said transduced stem cells to a host under conditions wherein at least some of said stem cells differentiate into cells of the type said cell type specific promoter is specific for (referred to, as differentiated stem cells); and
(c) allowing said differentiated stem cells to express said desired expressing a desired active or pharmaceutical agent.

13. A method of claim 12, wherein said host has a hormone deficiency condition or illness.

14. A method of claim 12, wherein the stem cells are selected from the group consisting of bone marrow derived stem cells, embryonic stem cells, adipose tissue derived stem cells, and cord blood cells.

15. A method of claim 13, wherein the condition is selected from the group consisting of type I diabetes, type II diabetes, hypogonadism, reproductive disorders, and obesity.

16. A method of claim 12, wherein the gene is selected from the group consisting of insulin, estrogen, testosterone, growth hormone, luteinizing hormone, follicle stimulating hormone, prolactin, leptin, and angiotensin.

17. The method of claim 12, wherein the tissue specific promoter is glucose-dependent insulinotropic polypeptide (GIP).

18. The method of claim 17, wherein the stem cells differentiate into K cells of the out.

19. The method of claim 12, wherein the stem cells are administered to the host by infusion into the superior mesenteric artery or celiac artery.

20. The method of claim 12 wherein the stem cells are further transduced with a killer gene under the control of a regulatable promoter, wherein the induction of the expression of the killer gene results in cell death of the cell expressing said gene.

21. The method of claim 20, wherein the killer gene is the fas ligand.

22. The method of claim 12, wherein the stem cells are administered to the host by injection into the intestinal mucosa.

23. Differentiated transduced stem cells delivered to the gut of a host for attaching to the gut and selectively expressing a desired active or pharmaceutical agent while engrafted in the intestine, said differentiated transduced stem cells comprising
(a) a DNA sequence containing a gene encoding said desired active or pharmaceutical agent, wherein said gene is operably-linked to a cell type specific promoter, and
(b) a cell type specific promoter which is specific for the differentiated transduced stem cells, wherein said differentiated transduced stem cells, while engrafted in the intestine, have the ability to express said desired active or pharmaceutical agent.

24. Differentiated transduced stem cells of claim 23, wherein said host has a hormone deficiency condition or illness.

25. Differentiated transduced stem cells of claim 23, wherein the stem cells are selected from the group consisting of bone marrow derived stem cells, embryonic stem cells, adipose tissue derived stem cells, and cord blood cells.

26. Differentiated transduced stem cells of claim 24, wherein the condition is selected from the group consisting of type I diabetes, type II diabetes, hypogonadism, reproductive disorders, and obesity.

27. Differentiated transduced stem cells of claim 23, wherein the gene is selected from the group consisting of insulin, estrogen, testosterone, growth hormone, luteinizing hormone, follicle stimulating hormone, prolactin, leptin, and angiotensin.

28. Differentiated transduced stem cells of claim 23, wherein the tissue specific promoter is glucose-dependent insulinotropic polypeptide (GIP).

29. Differentiated transduced stem cells of claim 28, wherein the stem cells differentiate into K cells of the out.

30. Differentiated transduced stem cells of claim 23, wherein the stem cells are administered to the host by infusion into the superior mesenteric artery or celiac artery.

31. Differentiated transduced stem cells of claim 23 wherein the stem cells are further transduced with a killer gene under the control of a regulatable promoter, wherein the induction of the expression of the killer gene results in cell death of the cell expressing said gene.

32. Differentiated transduced stem cells of claim 31, wherein the killer gene is the fas ligand.

33. Differentiated transduced stem cells of claim 23, wherein the stem cells are administered to the host by injection into the intestinal mucosa.

34. A population of transduced stem cells suitable for engrafting in the intestine of a host and differentiating therein once engrafted for selectively expressing a desired active or pharmaceutical agent comprising
a population of stem cells transduced with a DNA sequence containing a gene encoding a desired active or pharmaceutical agent, wherein said gene is operably-linked to a cell type specific promoter, and wherein at least some of said population of stem cells, once engrafted in the intestine of a host, have the ability to (a) differentiate into cells of the type for which said cell type specific promoter is specific and (b) express the desired active or pharmaceutical agent.

35. A population of transduced stem cells of claim 34, wherein said host has a hormone deficiency condition or illness.

36. A population of transduced stem cells of claim 34, wherein the stem cells are selected from the group consisting of bone marrow derived stem cells, embryonic stem cells, adipose tissue derived stem cells, and cord blood cells.

37. A population of transduced stem cells of claim 35, wherein the condition is selected from the group consisting of type I diabetes, type II diabetes, hypogonadism, reproductive disorders, and obesity.

38. A population of transduced stem cells of claim 34, wherein the gene is selected from the group consisting of insulin, estrogen, testosterone, growth hormone, luteinizing hormone, follicle stimulating hormone, prolactin, leptin, and angiotensin.

39. A population of transduced stem cells of claim 34, wherein the tissue specific promoter is glucose-dependent insulinotropic polypeptide (GIP).

40. A population of transduced stem cells of claim 17, wherein the stem cells differentiate into K cells of the out.

41. A population of transduced stem cells of claim 34, wherein the stem cells are administered to the host by infusion into the superior mesenteric artery or celiac artery.

42. A population of transduced stem cells of claim 34, wherein the stem cells are further transduced with a killer gene under the control of a regulatable promoter, wherein the induction of the expression of the killer gene results in cell death of the cell expressing said gene.

43. A population of transduced stem cells of claim 20, wherein the killer gene is the fas ligand.

44. A population of transduced stem cells of claim 34, wherein the stem cells are administered to the host by injection into the intestinal mucosa.

45. A pharmaceutical for engrafting in the intestine of a host and differentiating therein once engrafted for selectively expressing a desired active or pharmaceutical agent, said pharmaceutical comprising the population of transduced stem cells of claim 34, and a pharmaceutical excipient.

46. A pharmaceutical of claim 34, wherein said host has a hormone deficiency condition or illness.

47. A pharmaceutical of claim 34, wherein the stem cells are selected from the group consisting of bone marrow derived stem cells, embryonic stem cells, adipose tissue derived stem cells, and cord blood cells.

48. A pharmaceutical of claim 46, wherein the condition is selected from the group consisting of type I diabetes, type II diabetes, hypogonadism, reproductive disorders, and obesity.

49. A pharmaceutical of claim 45, wherein the gene is selected from the group consisting of insulin, estrogen, testosterone, growth hormone, luteinizing hormone, follicle stimulating hormone, prolactin, leptin, and angiotensin.

50. A pharmaceutical of claim 45, wherein the tissue specific promoter is glucose-dependent insulinotropic polypeptide (GIP).

51. A pharmaceutical of claim 45, wherein the stem cells differentiate into K cells of the out.

52. A pharmaceutical of claim 46, wherein the stem cells are administered to the host by infusion into the superior mesenteric artery or celiac artery.

53. A pharmaceutical of claim 45, wherein the stem cells are further transduced with a killer gene under the control of a regulatable promoter, wherein the induction of the expression of the killer gene results in cell death of the cell expressing said gene.

54. A pharmaceutical of claim 53, wherein the killer gene is the fas ligand.

55. A pharmaceutical of claim 45, wherein the stem cells are administered to the host by injection into the intestinal mucosa.

56. A pharmaceutical of claim 45, wherein the pharmaceutical excipient is a physiological buffer compatible with the transduced stem cells.

57. A pharmaceutical of claim 45, wherein the pharmaceutical excipient is a physiological saline compatible with the transduced stem cells.

58. A pharmaceutical of claim 45, wherein the pharmaceutical excipient is a glucose solution compatible with the transduced stem cells.

59. A pharmaceutical of claim 45, wherein the active or pharmaceutical agent is selected from the group consisting of a protein, peptide, enzyme, hormone, hormone synthesis enzyme, pro-drug and precursor.

60. A pharmaceutical of claim 45, wherein the active or pharmaceutical agent is selected from the group consisting of insulin, interferon, hormones, enzymes, somatostatin, anti-GIP, interleukins, chemokines, cytokines, EPO, nitiric oxide, synthetase, clotting factors, thrombin and pro-thrombin.
